(19) United States
(12) Patent Application Publication
(10) Pub. No.: US 2023/0289361 A1
(43) Pub. Date: Sep. 14, 2023

(54) METHOD FOR GENERATING ITEMS OF CONTEXT-DEPENDENT INFORMATION

(71) Applicant: SCHNEIDER ELECTRIC INDUSTRIES SAS, Rueil-Malmaison (FR)

(72) Inventor: Marco MENDES, Seligenstadt (DE)

(73) Assignee: SCHNEIDER ELECTRIC INDUSTRIES SAS, Rueil-Malmaison (FR)

(21) Appl. No.: 18/180,984

(22) Filed: Mar. 9, 2023

(30) Foreign Application Priority Data
Mar. 11, 2022 (EP) 22 161 598.2

(51) Int. Cl. G06F 16/25; G06F 16/2458; G06F 16/248

(52) U.S. Cl. CPC G06F 16/254; G06F 16/2462; G06F 16/248

(57) ABSTRACT

The invention relates to a method for generating items of context-dependent information for informing a user about a status of a technical system, wherein the items of context-dependent information of the technical system are provided by various data sources. In order to obtain items of information about a new context of the technical system without real data sources, it is proposed that estimated context-dependent items of information of a new context are generated by correlating the data elements of the various data sources and without knowledge of real values of the new context, but based on known and experience-related values, and provided to the user.

The invention relates to a method for generating items of context-dependent information for informing a user about a status of a technical system, wherein the items of context-dependent information of the technical system are provided by various data sources.

Context-dependent systems are known from the prior art, which use information about their context, i.e., their environment, in order to adapt their behavior thereto. The basis on which such systems work are items of information provided by a wide variety of data sources or sensors. The determined context is used to adapt the behavior of the system, in particular the behavior of a user interface for informing a user. Context is defined here, for example, as any kind of information that can be used to characterize a situation of an entity in interaction with other entities (Anind K. Dey, et al.: Towards a Better Understanding of Context and Context-Awareness. Graphics, Visualization and Usability Center and College of Computing, Georgia Institute of Technology, Atlanta (Georgia), Jul. 8, 1999)

Various information contexts are available in automation systems, which are not “linked” to one another, however. In this context, for example, OPC UA “life” data of an automation device are to be mentioned, which may not have a direct relationship to a location of the automation device, since the various information models used have no “contact”.

Another example is the diagnosis of, for example, automation devices, wherein a large number of information contexts have to be taken into consideration, for example location of the automation device, topology, current application, and adjacent automation devices, in order to be able to present much more precise results.

The automatic processing of items of information is currently made more difficult by the fact that items of information are separated and semantic aspects are missing; because the items of information obtained from the diagnosis have to be “understood” by computing devices, i.e., the semantic interoperability and the way in which data are connected to each other has to be improved.

There is also often the problem that not all the required contexts are available. Examples are items of environmental information about the location or room at or in which an automation device is installed if it has no sensors. When a context is not modeled or when data are not available, it would be advantageous to be able to provide “estimated” or “approximate” items of information, i.e., items information derived from experience without exact measurement

Methods are also known from the prior art which work with semantic rules on the basis of rule engines (semantic rule engine SRE). Semantic rule engines enable the implementation of dynamic and flexible rule-based strategies and can process queries and supply results by deriving additional knowledge from terms defined in ontologies.

While semantic rule engines can be used to define actions, no rules have been generated up to this point for “estimated” items of information. However, semantic rule engines are used as a kind of data source, for example to provide a location context and relationships derived therefrom, wherein OPC UA data are linked to the location. However, the strategy for this assignment is predetermined.

So far, no solution is known for using a semantic rule engine as a data source for “estimated” or “approximate” items of information.

With OPC Unified Architecture (OPC UA), a standard for data exchange is known as a platform-independent, service-oriented architecture. As the latest generation of all specifications of the Open Platform Communications (OPC) from the OPC Foundation, OPC UA differs significantly from its predecessors, in particular by the ability not only to transport machine data (control variables, measured values, parameters, etc.) not only to transport, but also to describe them semantically in a machine-readable manner. Therefore, OPC-UA, or any other device access and device description technology, is capable of modeling items of device information. However, OPC-UA has so far been used as an information source, similar to semantic rule engines, for example to provide a topology context and a diagnosis context.

Current items of information are provided for the diagnosis of an automation device, for example a speed controller, e.g., error/warning code, supporting items of information/statuses, service notifications, probe cause, remedy, delete error/warning code, error/warning history, test sequences, and warning groups. However, many of the provided items of information are mostly device-specific. Furthermore, items of information about the probe cause and remedy are statically defined in the device information model. In addition, further items of context information, e.g., other devices, systems, and environments, are not taken into consideration in order to provide more precise and comprehensive items of diagnostic information.

Proceeding from this, the present invention is based on refining a method of the type mentioned at the outset such that the above-mentioned problems of the prior art are eliminated. In particular, items of information about a new context of the technical system are to be generated without real data sources.

The object is achieved according to the invention by a method having the features of claim 1, wherein items of estimated context-dependent information of a new context are generated by correlating the data elements of the various data sources and without knowledge of real values of the new context, but based on known and experience-related values, and provided to the user.

The invention thus relates to a method for deriving new contexts of estimated or approximate items of information for industrial automation devices in order to support or advise a user, based on assumptions, existing contexts, their relationships, and previous experiences.

The method is based on existing contexts (data sources) and their relationship to one another, which can be referred to as contextualizing data.

The new contexts are not to be considered as a direct/concrete data source or information source, but are derived based on existing items of device information and previous events or user experiences.

The method according to the invention offers the possibility of deriving new knowledge from existing knowledge in the form of, for example, diagnostic data, topology data, location data, or device data in order to support an operator with various possible solutions for a specific problem.

The items of context-dependent information can preferably be provided as information models in the form of device-specific data, e.g., device data, diagnostic data, and/or measurement data, by first data sources in the form of devices of automation technology, such as OPC UA servers.

Furthermore, in addition items of context-based information can preferably be provided as information models in the form of semantic data by second data sources in the form of ontologies, such as device ontology, location ontology, diagnostic ontology, by an ontology/inference machine.

The items of context-based information are preferably represented as data elements in the information models, wherein a context ID is assigned directly or indirectly to each data element so that a specific concept, a specific property, or a specific device is actually represented identically in the various data sources.

The assignment of the context ID to the data element preferably takes place in an OPC UA information model by way of a namespace or a node ID, wherein an assignment table is used to convert the namespace or the node ID into the context ID.

A preferred procedure provides that the assignment of a context ID to a data element in an ontology is modeled for each instance.

A database, in particular a TimeSeries database, can preferably be used as the data source for current and past values.

The correlation of the items of context-based information from the various data sources preferably comprises the following steps:
establishing a connection between a data binder unit and the data sources,
assigning between items of context-based information by contextualizing OPC UA data, in that context IDs of OPC UA nodes are received with equivalent IDs in the information models of the ontologies,
dynamically creating a database based on incoming items of information, wherein the context IDs are compared to classes of the ontologies and a new relationship is generated in the database upon correspondence.

Generating the new context and associated data elements particularly preferably comprises the following steps:
a) triggering an event, for example changing a diagnostic value or status query of the user
b) retrieving current device data or reading them from the TimeSeries database
c) possibly updating the data correlation
d) triggering a query at a semantic rule engine to obtain inferences about known contexts or known items of information
e) creating the new context of items of estimated context-based information based on context rules and based on the result of the semantic rules in step d)
f) assigning the first query to the known and estimated items of information and formulating a result.

The provision of the new context of items of estimated context-dependent or context-based information preferably includes the presentation of semantic information by a user interface.

Items of context-dependent diagnostic information are preferably generated based on the items of reliable context-dependent information and the items of estimated context-dependent information.

The main use case is preferably the diagnosis of the functional status of devices, in particular automation devices, in connection with the generation of new contexts that are used to inform a user about possible situations.

When diagnosing devices with context, for example, possible associations between the devices are also reported. For example, if some devices have a poor functional condition and are located in the same room, the user can receive a notification about those devices, including location, with suggestions of possible environmental conditions, which represents the new context.

For example, if both devices have a high operating temperature and are located in the same room, an additional recommendation based on the above-described context can be to also check the room environment, as this can result in the problem described. In this case, a new context “room having a variable temperature” is generated, the diagnosis of which indicates a malfunction, for example of the ventilation.

The diagnostic function of devices and systems will be improved by the method according to the invention. The invention not only results in an improved diagnosis but could also be applied to other cases, such as conditional monitoring and multiple configuration.

However, diagnosis is not the only application for the method according to the invention.

The generation of context-dependent services can be viewed as a virtual sensor. The generation of context-dependent services is based on the generation of an estimated or approximate context, for example items of estimated environmental information, which contain assumptions, various data sources, and experiences from the past, but no real sensor values.

The estimated contexts or virtual sensors can be created as follows:
1. Establish connection to data sources (contexts),
2. Correlate different contexts with one another, wherein unique identifications (IDs) are used for the same concept in different data sources.
3. Generating new estimated contexts and associated data elements in response to events,
   a) Search for registered events that are triggered (for example a bad diagnostic value from a device or a query).
   b) Call up associated context (for example diagnosis) from that event and execute a semantic query for this context, wherein all instructions are output that are assigned to this context.
   c) Check instructions on the basis of context rules, wherein if a rule corresponds, an estimated context and a virtual sensor value or other action is generated, which is assigned to this and wherein it is to be noted in particular that context rules are based on earlier experiences and assumptions.
4. Output instructions to the user

A particularly preferred embodiment relates to a method for generating items of context-dependent information for informing a user about a state of a technical system, wherein the items of context-dependent information of the technical system are provided by various data sources and wherein items of context-dependent information of a new context are generated and provided to the user. It is provided that that estimated items of context-dependent information of a new contextthat does not represent a real data source are generated by correlating data elements of the various data sources, but based on known and experience-related values,

wherein the generation of the estimated items of context-dependent information of the new context and associated data elements comprises the following steps:
g) triggering an event
h) retrieving current device data
i) possibly updating the data correlation
j) triggering a query at a semantic rule engine to obtain inferences about known contexts or known items of information
k) creating the new context of items of estimated context-dependent information based on context rules and based on the result of the semantic rules in step d)
l) assigning the query to the known and estimated items of information and formulating a result.

Another preferred embodiment relates to a method for acquiring data for diagnosing devices in an automation system, comprising:
modeling and providing first items of context information in the form of device-specific data such as device data, diagnostic data, and/or measurement data by first data sources in the form of the devices,
modeling and providing second items of context information in the form of semantic data of the devices by second data sources in the form of ontologies such as device ontology, location ontology, diagnostic ontology, and/or in the form of information models such as OPC UA topology,
defining requests, invents, and instructions which are assigned to the items of context information,
defining context-related rules,
establishing a connection to the various first and second data sources (contexts),
correlating the various data sources (contexts) with one another, wherein unique context IDs are used for identical concepts in various data sources,
acquiring an event, such as triggering a registered event, for example a setpoint value exceeding a diagnostic value,
searching for a context (for example diagnosis) associated with the event,
carrying out a semantic query for the acquired context in order to obtain all instructions linked to the context,
checking the instructions based on context-related rules, and
generating a new item of estimated context-based information (diagnostic information) and a corresponding data element (virtual sensor value) when one of the context-related rules is followed.

The invention thus relates to a method for creating new contexts of items of estimated information for industrial automation devices, in particular if no data or sensors are available for this context. According to the invention it is provided that the new contexts will be derived from existing contexts, e.g., from diagnostic data, topology data, location data of the devices, the independent items of context information will then be linked to one another to assist an operator with different possible solutions for a specific problem.

The various items of context information and their associations provide a new kind of data, i.e., the generation of estimated items of information based on assumptions, especially when querying and analyzing data for conclusions and expertise.

According to the invention, requests, events, and instructions assigned to the items of context information are defined. By defining a request, an event, or an instruction, it is registered. Every registered event has a context ID and using the context ID, it is possible to acquire the detailed context by searching for it in the ontology database. The context is consequently acquired using context IDs.

The list of registered events is part of the program memory. Correlation of the data by means of the data binder is a function of the program so that data sets can be linked to one another if a query is made. The context ID is used for this The context-related rules are stored in a file that is read by the program. However, the rules can also be part of the program.

A semantic query is carried out for the acquired context in order to obtain all instructions linked to the context. These queries are carried out in the ontology database. A correlation is just a program function. The registered events accordingly have a context ID and corresponding semantic entries in the ontology database also have context IDs. In this way, it is possible to search for and find the instructions.

The new contexts are generated based on context-related rules. If a rule is TRUE, the new context is generated, wherein the new context is described in the rule. The new contexts can also be part of the sequence of the program and thus part of the program memory.

According to the invention, estimated items of context-dependent information of a new context are generated without knowledge of real values of the new context, i.e., the items information about a new context are generated without a real data source. For example, the ACTUAL temperature of a virtual sensor is not a real value, but the value can be estimated. The new context can thus be the temperature of a room, even though there is no “real” sensor and corresponding value in the room.

The system comprises at least two databases, namely the TimeSeries database and the ontology database.

Other details, advantages, and features of the invention result not only from the claims, the features to be inferred therefrom as such and/or in combination, but also from the following description of a preferred exemplary embodiment to be inferred from the drawings.

In the figures:

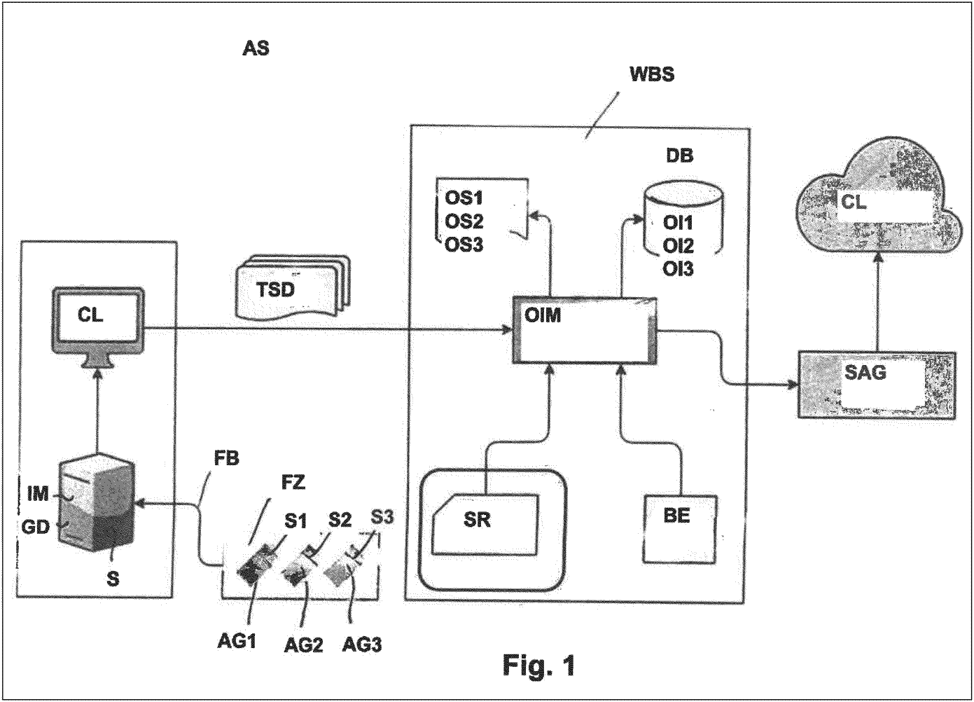

FIG. 1 shows a schematic representation of an automation system having a knowledge-based system,

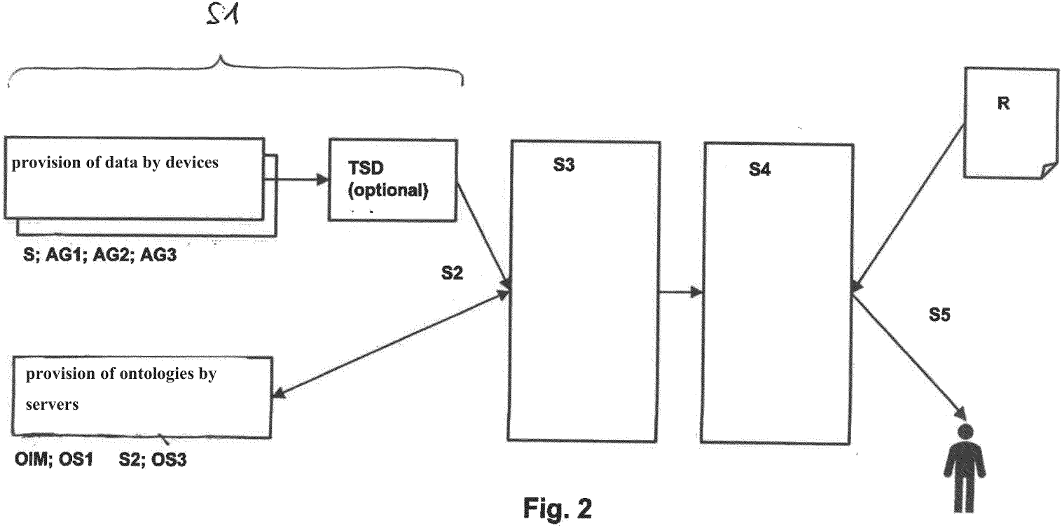

FIG. 2 shows a schematic representation of the method according to the invention,

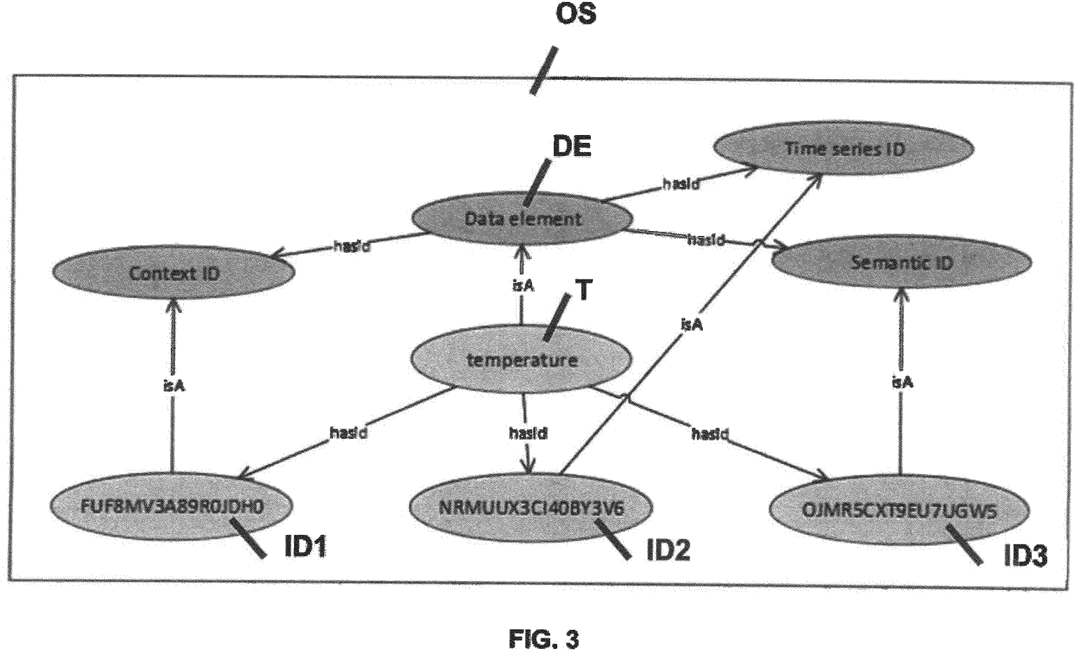

FIG. 3 shows a schematic representation of an ontology scheme for describing a data element,

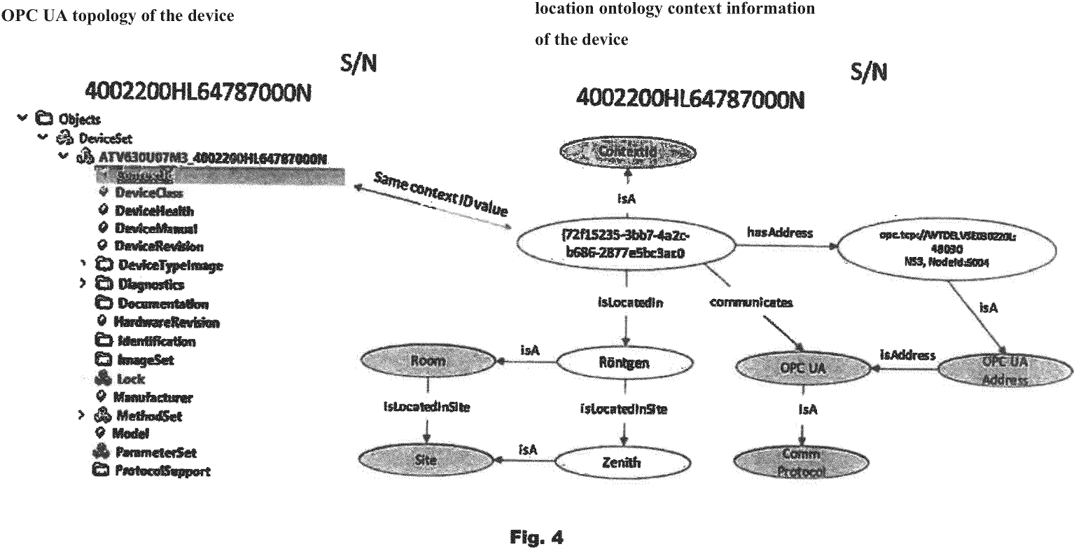

FIG. 4 shows a schematic representation of an ontology scheme for describing a location of a device,

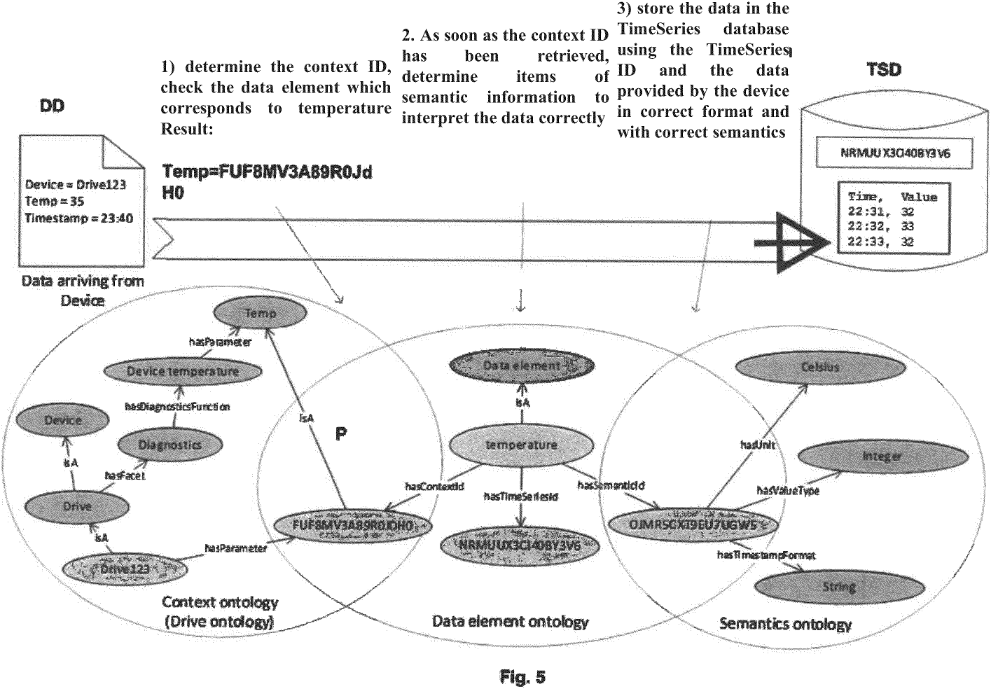

FIG. 5 shows a schematic representation of the “binding” of different information contexts,

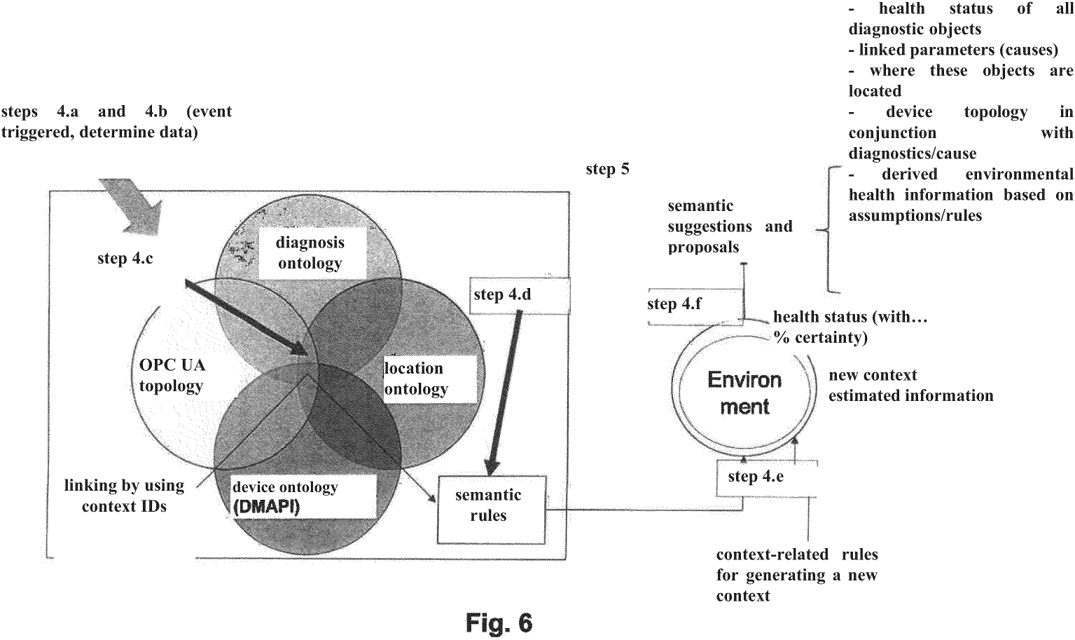

FIG. 6 shows a schematic representation of method steps for generating a new information context, and

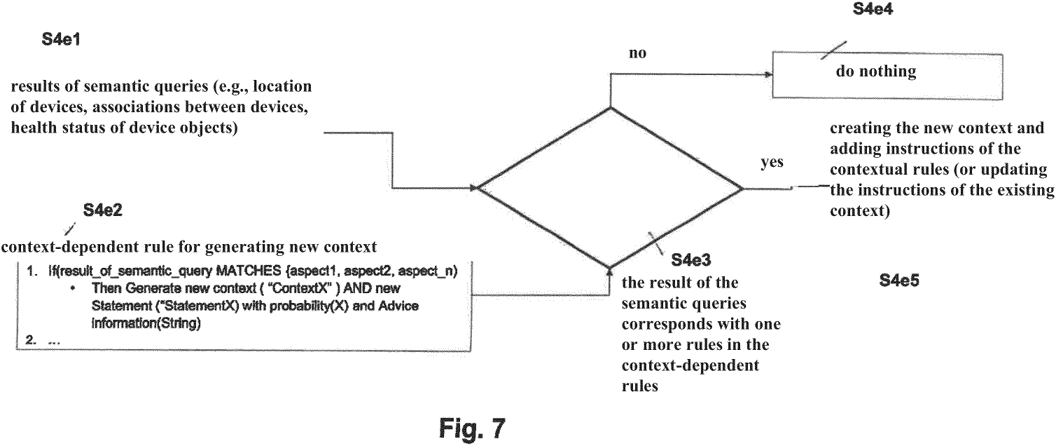

FIG. 7 shows a schematic representation of method steps for applying context-dependent rules.

FIG. 1 schematically shows an automation system AS having devices AG1, AG2, AG3 of automation technology in the form of, for example, drive, speed controller, programmable logic controller, sensor, such as temperature sensors, current sensors, voltage sensors, etc., each representing data sources as such. The devices AG1, AG2, AG3 are, for example, arranged together in a manufacturing cell FZ and are connected via a field bus FB, such as Modbus, to a server S, preferably an OPC UA server. The server S is connected to a knowledge-based system WBS via a client CL, preferably an OPC UA client.

The knowledge-based system WBS includes an ontology/inference engine OIM, which processes rule knowledge and factual knowledge. The rule knowledge is provided in the form of semantic rules SR. The factual knowledge is provided by data sources in the form of the server S, the device data GD of the devices via the client CL, and in the form of ontologies OS1, OS2, OS3 and associated ontology instances OI1, OI2, OI3, which are stored in databases DB. An operator can send queries to the ontology/inference engine OI M and receive instructions therefrom via an operating unit BE. In addition, the ontology/inference engine OIM can be coupled to an external data source CL, such as a cloud, via a software agent SAG.

The server S is a data source that provides information models IM of the device data GD of the connected devices AG1, AG2, AG3. The information models IM provide build and runtime data in the form of device parameters and their relationships to one another (topology). Current data of the devices AG1, AG2, AG3 are provided via the client CL.

In order to avoid a continuous pool of data from the server S, the data can optionally be temporarily stored in a database, for example a TimeSeries database TSD, which can then be used as another data source for current and historical data values.

Additional models can be defined with the aid of the ontologies OS1, OS2, OS3. The ontologies OS1, OS2, OS3 are used to describe data models of certain information domains. An information domain is, for example, a diagnosis, a location, or a device. The ontologies OS1, OS2, OS3 represent data sources. Depending on the application, examples of ontologies are a diagnosis ontology OS1, a location ontology OS2, and a device ontology OS3. Items of information are supplemented with semantic descriptions by the ontologies OS1, OS2, OS3, so that these items of information can be interpreted by the ontology/inference engine OIM. The device ontology OS3 contains, for example, known data about devices, such as device descriptions, IP addresses, communication parameters, etc.

FIG. 2 shows, solely schematically, method steps of the method according to the invention.

In a step S1, different context-based or context-dependent items of information are modeled. The devices AG1, AG2, AG3 provide device-specific data GD, such as device data, diagnostic data, and/or measurement data, via the OPC UA server S.

Furthermore, semantic data of the devices and/or the system are modeled. The semantic data extend the information about the devices and/or the system. The semantic data are provided via an information model IM, such as OPC UA topology, or the ontologies OS1, OS2, OS3, such as location topology.

The modeling of the different context-dependent items of information depends strongly on which devices AG1, AG2, AG3 or server S are used as the data source and which context-dependent items of information are required. For OPC UA, each server S provides an information model IM that primarily represents build and runtime data of the devices, for example parameters, and their relationship to one another in the form of a topology.

Additional models are defined using the ontologies OS1, OS2, OS3 and are made available by the ontology/inference engine. The ontology/inference engine is also configured to carry out queries.

In a step S2, a connection is established to the data sources in the form of the devices AG1, AG2, AG3, the server S, and/or the ontologies OS1, OS2, OS3.

In a step S3, the various data sources are “linked”, i.e., the data are contextualized, which is explained in detail hereinafter.

In a step S4, new contexts are generated and associated data elements are generated as a response to queries or events.

In a step S5, a user is informed or advised on the basis of the new contexts and associated data elements.

The basic requirement for the method according to the invention is that a direct or indirect context ID is assigned to each data element in the information models and the ontologies.

In OPC UA models, the namespace and the node ID, which uniquely identify a data element, can be used for this purpose. An assignment table can be used to convert a namespace or node ID into a context ID.

In the ontologies OS1, OS2, OS3, the assignment has to be modeled in each ontology instance OI1, OI2, OI3, for example by using the relationship “hascontext-ID” or “hasID” as shown in FIG. 3.

FIG. 3 shows an ontology scheme OS of the term temperature T. The temperature T is defined as a data element DE via the relation “isA”. Furthermore, an identifier ID1, ID2, ID3 is assigned to the term temperature T via the relations “hasId”. The identifications ID1, ID2, ID3 are defined via the relations “isA” as context ID, time series ID, and semantic ID. The context ID, time series ID, and the semantic ID are also assigned to the data element DE via the “hasId” relations.

In this way, each data element in the information model is assigned a direct or indirect context ID.

Once the information models and/or ontologies are provided by the different data sources as described in step S1, a connection to the different data sources has to be established. This is done by means of a data binder. Device data are retrieved, for example, directly from the OPC UA server S or indirectly from the TimeSeries database TSD. Items of location information can be obtained, for example, by querying the ontology/inference engine.

Querying of data or items of information and merging of data or items of information are only carried out when there are events for generating or updating the context-dependent items of information. For example, the generation of new contexts is to be updated every time the value of a function status object changes.

Once the connection to the individual data sources is established, the different data sources have to be connected to one another. This means that a specific term, property, or device is actually the same in the different data sources. This is carried out with the aid of the preferably identical context IDs assigned to the terms, properties, and devices according to FIG. 3.

FIG. 4 shows an OPC UA topology of a device having the serial number 4002200HL64787000N and context-dependent items of information of the device with the same serial number in the form of a location ontology. For example, a device exists in an OPC UA address space, for example speed controller ATV630XXX, having a specific context ID. The same context ID is also used in the location ontology shown. This means that both terms or devices are actually the same, but are described differently depending on the data source; because the OPC UA information model provides items of topology and diagnostic information of the device and the location topology provides items of location information for the same device.

FIG. 4 furthermore shows that the same value for the context ID is used in the OPC UA topology and in the location topology. In the location ontology, this value is assigned to the term “context ID” via the “isA” relation. Furthermore, the term “Röntgen” is assigned to the value via the “isLocatedIn” relation. The term “Röntgen” is assigned to the term “Room” via the relation “isA”. Furthermore, the term “Zenith” is assigned to the term “Röntgen” via the relation “isLocatedInSite”. The term “Zenith” is assigned to the term “Site” via the relation “isA”.

The data binder is designed to maintain the association between items of context-dependent information from different data sources. A context update of data provided via OPC UA, for example, can be achieved by incorporating context IDs in relevant OPC UA nodes with equivalent identifications in the ontology models. This makes it possible to connect or link a topology context, as provided by OPC UA, with other contexts modulated by ontologies. The same can also be applied to the storage of TimeSeries data.

Such a database having context-updated data is created dynamically based on the incoming items of information, wherein the context IDs are compared to the ontology classes. If they correspond, a new relationship is generated in the database. If an instance already exists, a new value is updated, for example in the TimeSeries database. The comparison of incoming data with classes in the ontology can be done via REGEX expressions, for example.

FIG. 5 shows an example of the generation of items of information based on interconnected contexts. Device data DD are provided by the device AG1, for example a drive, for example “Device = Drive 123”, “Temp = 35” and “Timestamp = 23:40”. It is known from the device ontology that the device “Drive 123” is a drive. The drive is a device. The drive also has a sub-aspect “Diagnostics” with the function “Device temperature” having the parameter “temp”.

The device “Drive 123” has a parameter “P” which represents a temperature. A context ID is then assigned. A check is made using a data element that corresponds to the parameter “temp”. This is carried out using a data element ontology, wherein the term “temperature”, which is a data element, is assigned the parameter “P” as a context ID via the relation “hasContextId”. Furthermore, a time series ID and a semantic ID are assigned to the term “temperature”.

Once the context ID has been retrieved, semantic items of information have to be retrieved in order to know how to correctly interpret the data. A semantic ontology is used for this. A unit “celsius”, a value type “integer”, and a time stamp format “string” are assigned to this term via the semantic ID of the term “temperature”.

The data are then stored in the TimeSeries database using the TimeSeries ID and the data provided by the device in the correct format and correct semantics.

FIG. 6 shows in detail method steps for generating new contexts and associated data elements as a reaction to an event (step S4). The generation comprises the following steps:

S4a) An event is triggered. An event can be the change of a diagnostic condition value exceeding a setpoint value or the query of a user requesting status.

S4b) Current device data are retrieved or read from the time series database TSD.

S4c) If necessary update the data binding according to step S3.

S4d) Trigger a query to the ontology/inference engine (semantic rule engine) in order to draw conclusions about specific contexts or information. After this step, all necessary contexts of safe or actual information are available.

S4e) Generate new contexts based on context-dependent rules and based on the result of the semantic rules in step 4d).

S4f) Assign the original request to the secure and estimated items of information and formulate a final result.

FIG. 7 shows, solely schematically, method step S4e of generating new contexts based on the results of a semantic query S4e1 and applying context rules R according to step S4e2.

The semantic query according to step S4e1 provides, for example, the location of the device, relationships between devices, and the functional status of objects of the device.

In method step S4e2, context-related rules R are defined in order to generate new contexts. One rule reads, for example, “if (result_semantic_query MATCHES {aspect1, aspect2, aspect_n}) Then generate new context (“ContextX”) AND new Statement (“StatementX”) with probability(X) and Advice information (“string”)″.

In step S4e3 it is checked whether the result of the semantic queries corresponds to one or more rules R of the context-related rules R. If “no”, nothing is done in step S4e4. If “yes”, in step S4e5 a new context with data element is generated from the context-related rules or instructions of already existing contexts are updated.

A possible scenario is, for example, if two or more devices are in the same room (aspect) and devices have a thermal diagnostic object with status > 0 (aspect); then generate new context (“environment”) and instruction “health” = 1 with probability of 50%. Advice information = “Please check room environment first”.

Method step S4 is to be explained in more detail hereinafter in an example:

1. Define a set of events: E = {E₁, E₂...Eₙ}.

An event can be a change of a diagnostic value from 0 (okay) to 4 (outside specification), or a query by a user, etc.

Every event e E E has one or more assigned contexts Ce = {C₁, C₂...Cn}. For example, the thermal diagnosis of a device is in the context “Diagnosis”.

Every event e E E has one or more assigned values Aₑ = {a₁, a₂...a_{N}}. For simplicity, the values assigned to an event are all other values in the same context as e. For example, if the context of e is “Diagnosis”, all other diagnostic values are considered assigned values, as well as their first-order relationship, which is the cause value linked to each diagnostic object. For example, there is a cause for a thermal diagnostic object, which is the operating temperature.

2. If one or more events e E E change, acquire their current value and the current value of all assigned values A of all e E E that have changed. If necessary, these values have to be updated in the TimeSeries database TSD.

If the value for the thermal diagnosis has changed, for example, from 0 to 4, the new value (4) has to be retrieved and the value of all other diagnostic objects in all devices has to be updated in the TimeSeries database.

3. Create a semantic query for all contexts Cₑ assigned to e, wherein e is the event that has changed. A possible query is: “search C”.

In the example with diagnosis, a combined context “diagnosis” is used, which “connects” the location ontology and the OPC UA topology in a single context.

The query “search C” will return a series of instructions S = {S₁, S₂...S_{N}} for this context C. An instruction is a direct or indirect (derived) relationship of type “diagnosis”. First-order relationships return all objects that are of the type “diagnosis”, such as “Thermal” “isA” “diagnostics”, “ATV360xxx” “contains” “Thermal”, “Thermal” “contains” “HealthStatus”, “HealthStatus” “hasA” “Value”, “Value” “is” “4”, “ATV630xxx” “isLocated” “FactoryCell2”, “ATV 630xxx” “isA” “Device”.

4. Check whether the previous statements s E S of the query correspond to one or more context-based rules R = {R₁, R₂...R_{N}}. A rule is an algorithm that takes as input the set of statements from the query result S and returns true or false if S corresponds to the rules expressed by the algorithm.

### EXAMPLE

Rule r(S) => “IF two or more devices are in the same room (same X in “islocated” X) AND devices have a thermal diagnostic object (“Thermal” “isA” “Diagnostics”, X “contains” “Thermal ”) with HealthStatus value > 0; THEN RETURN TRUE”.

5. If one or more rules r in R are TRUE, then carry out the associated actions with the matching rules. A typical action would be to generate a new context with one or more properties and an associated probability.

### EXAMPLE

Generate new context (“environment”) AND instruction “health” = 1 with probability of 50%. Items of advice information = “Please check room environment first”.

Step S5 of the method relates to user advice.

The advice of the user depends strongly on the user interface. Basically, the items of information of the existing and new contexts are formatted in such a way that the user can understand them. An adviser information string can be used here as an item of information for the user. A “user” can also be an additional computing system that can understand the result of the query semantically.

The method according to the invention allows new contexts of items of estimated information for industrial automation devices to be generated, even if no data or sensors are available for these contexts. The new contexts are derived from existing contexts such as diagnostic data, topology data, or location data of the devices. Then the independent items of context-dependent information are connected to one another. An operator is then assisted with various possible solutions to a given problem by instructions.

The various items of context-dependent information and their assignment provide another value level for data, i.e., the generation of estimated items of information based on assumptions, in particular when querying and analyzing data for conclusions and expertise.

In this context, the functional status (health status) of a thermal object of an automation device, such as a speed controller, should also be considered, which is arranged in a production cell in which another automation device is also arranged that has the same status. Both automation devices are connected to a SCADA (Supervisory Control and Data Acquisition) system via OPC UA and are used in a process controller to control several motors. It would be very helpful for a user to receive the items of information about the status of the thermal objects of the automation devices as soon as there is a functional problem.

The application of the method according to the invention in relation to an environmental context is to be described with reference to FIG. 1 using an example. The automation devices AG1, AG2, AG3 are located together in the manufacturing cell FZ. An environmental context does not exist since no real sensor or other real value is available that provides information about the status of the manufacturing cell FZ. There is no possibility to acquire the status, for example the temperature, in the manufacturing cell FZ.

However, the location of the devices AG1, AG2, AG3 is known and there is the possibility of acquiring the operating temperature of the devices AG1, AG2, AG3 via device sensors S1, S2, S3.

This raises the question of whether, on the basis of the measured temperatures, assumptions can be made about the condition or the environment of the manufacturing cell FZ based on the existing values, here temperature values of the devices.

This is where the invention comes in.

1. In a first step, a connection is established to the data sources (contexts) OPC UA server S and the location ontology. The OPC UA server provides items of OPC UA information that supply the temperature values of the device. The location ontologies provide items of location information of the devices AG1, AG2, AG3.

2. The different contexts are correlated or bound to one another, wherein unique context IDs are used for the same terms in different data sources.

New estimated contexts and associated data items are then generated as a response to queries or events.

3.1 Search for registered events that are triggered, for example at least one increased temperature value.

3.2 Query the assigned context, for example diagnosis, of the event and carry out a semantic query for this context. All statements assigned to this context are returned. For example: “Thermal” “isA” “diagnostics”, “ATV360xxx” “contains” “Thermal”, “Thermal” “contains” “HealthStatus”, “HealthStatus” “hasA” “Value”, “Value” “is” “4”, “ATV630xxx” “isLocated” “FactoryCell2”, “ATV 630xxx” “isA” “Device”.

3.3 Check statements based on context rules. If a rule corresponds, generate estimated context and a virtual sensor value or other action assigned to this rule. The context rules are based on previous experience and assumptions. For example, one rule is:

IF two or more devices are in the same room (same X in “isLocated” X) AND the devices have a diagnostic object temperature (“Temperature” “isA” “diagnostics”, X “contains” “Temperature”) with value ! = OK ; THEN generate new context (“Environment”) AND instruction “Temp” = NOK with certainty of 50%. Items of advice information = “Please check room environment first”.

1. A method for generating items of context-dependent information for informing a user about a status of a technical system, wherein the items of context-dependent information of the technical system are provided by various data sources,
wherein estimated context-dependent items of information of a new context are generated by correlating the data elements of the various data sources and without knowledge of real values of the new context, but based on known and experience-related values, and provided to the user.

2. The method as claimed in claim 1, wherein the items of context-dependent information are provided as information models in the form of device-specific data, e.g., device data, diagnostic data, and/or measurement data, by first data sources in the form of devices of automation technology, such as OPC UA servers (= Open Platform Communications Unified Architecture servers).

3. The method as claimed in claim 1, wherein the context-based items of information are provided as information models in the form of semantic data by second data sources in the form of ontologies, such as device ontology, location ontology, diagnostic ontology, by an ontology/inference machine.

4. The method as claimed in claim 2, wherein the items of context-based information are represented as data elements in the information models, wherein a context ID is assigned directly or indirectly to each data element so that a specific concept, a specific property, or a specific device is actually represented identically in the various data sources.

5. The method as claimed in claim 4, wherein the assignment of the context ID to the data element takes place in an OPC UA information model by way of a namespace or a node ID, wherein an assignment table is used to convert the namespace or the node ID into the context ID.

6. The method as claimed in claim 4, wherein the assignment of the context ID to the data element in an ontology is modeled for each instance.

7. The method as claimed in claim 1, wherein a database, in particular a TimeSeries database, is used as the data source for current and past values.

8. The method as claimed in claim 4, wherein the correlation of the items of context-based information from the various data sources comprises the following steps:
establishing a connection between a data binder unit and the data sources,
assigning between items of context-dependent information by contextualizing OPC UA data, in that context IDs of OPC UA nodes are received with equivalent IDs in the information models of the ontologies, and
dynamically creating a database based on incoming items of information, wherein the context IDs are compared to classes of the ontologies and a new relationship is generated in the database upon correspondence.

9. The method as claimed in claim 1, wherein generating the new context and associated data elements comprises the following steps:
a) triggering an event, for example changing a diagnostic value or status query of the user
b) retrieving current device data or reading them from the TimeSeries database
c) possibly updating the data correlation
d) triggering a query at a semantic rule engine to obtain inferences about known contexts or known items of information
e) creating the new context of items of estimated context-dependent information based on context rules and based on the result of the semantic rules in step d)
f) assigning the first query to the known and estimated items of information and formulating a result.

10. The method as claimed in claim 9, wherein the provision of the new context of estimated items of context-dependent information comprises the presentation of semantic information by a user interface.

11. The method as claimed in claim 9, wherein items of context-dependent diagnostic information are generated based on the items of reliable or known context-dependent information and the items of estimated context-dependent information.

12. A method for acquiring data for diagnosing devices in an automation system, comprising:
modeling and providing first items of context information in the form of device-specific data such as device data, diagnostic data, and/or measurement data by first data sources in the form of the devices,
modeling and providing second items of context information in the form of semantic data of the devices by second data sources in the form of ontologies such as device ontology, location ontology, diagnostic ontology, and/or in the form of information models such as OPC UA topology,
defining requests, events, and instructions which are assigned to the items of context information,
defining context-related rules,
establishing a connection to the various first and second data sources (contexts),
correlating the various data sources (contexts) with one another, wherein unique context IDs are used for identical concepts in various data sources,
acquiring an event, such as triggering a registered event, for example a setpoint value exceeding a diagnostic value,
searching for a context (for example diagnosis) associated with the event,
carrying out a semantic query for the acquired context in order to obtain all instructions linked to the context,
checking the instructions based on context-related rules, and
generating a new item of estimated context-based information (diagnostic information) and a corresponding data element (virtual sensor value) when one of the context-related rules is followed.
